(19) United States
(12) Patent Application Publication
(10) Pub. No.: US 2023/0251195 A1
(43) Pub. Date: Aug. 10, 2023

(54) IDENTIFYING FLUORESCENCE CONTRIBUTIONS OF MULTIPLE FLUORESCENT COMPOUNDS IN A SAMPLE

(71) Applicant: Vanderbilt University, Nashville, TN (US)

(72) Inventors: Nicholas M. Adams, Nashville, TN (US); Frederick R. HASELTON, Nashville, TN (US); Zackary A. ZIMMERS, Nashville, TN (US)

(21) Appl. No.: 18/164,028

(22) Filed: Feb. 3, 2023

Related U.S. Application Data
(60) Provisional application No. 63/306,575, filed on Feb. 4, 2022.

(51) Int. Cl. G01N 21/64; C12Q 1/6858

(52) U.S. Cl. CPC G01N 21/6428; C12Q 1/6858; G01N 2021/6439

(57) ABSTRACT

The number of unique testable conditions that can be detected in parallel from a sample in a PCR assay can be increased without need for larger sample volumes or additional expensive instruments. At least two fluorescent compounds, each designed to have a peak fluorescence excitation and/or emission within a common spectral channel, can be added to a sample in a tube, wherein each of the at least two fluorescent compounds is used to test for a unique testable condition. PCR assay can be performed on the contents of the tube. Then a spectral channel signal of each of the at least two fluorescent compounds can be determined based on fluorescence emission measurements and at least two properties of each of the at least two fluorescent compounds, wherein the spectral channel signals are analyzed to indicate a presence or absence of each of the unique testable conditions.

### CROSS-REFERENCE TO RELATED APPLICATIONS

This application claims the benefit of U.S. Provisional Application Ser. No. 63/306,575, filed 4 Feb. 2022, entitled “IDENTIFYING FLUORESCENCE CONTRIBUTIONS OF MULTIPLE FLUORESCENT COMPOUNDS IN A SAMPLE”. The entirety of this application is incorporated by reference for all purposes.

### GOVERNMENT SUPPORT

This invention was made with government support under grant number R01 AI 157827 awarded by National Institutes of Health. The government has certain rights in the invention.

### TECHNICAL FIELD

The present disclosure relates generally to identifying fluorescence contributions of multiple fluorescent compounds in a sample and more specifically, to methods for increasing the number of distinct testable conditions that can be detected in parallel from a single sample by utilizing unique properties of fluorescent compounds that have peak excitations and/or emissions in a common spectral channel.

### BACKGROUND

Many clinically relevant pathogens present with similar symptoms. For example, symptoms of respiratory infection (e.g., cough, fever, congestion, shortness of breath) are associated with bronchitis, bronchiolitis, pneumonia, and the common cold, which are caused by various virus and bacteria strains, including SARS-CoV-2, influenza A and B, respiratory syncytial virus (RSV), metapneumovirus, parainfluenza, rhinovirus, Chlamydia pneumoniae, or Mycoplasma pneumoniae. Patient treatment and management decisions vary based on the specific pathogen causing the common symptoms. Identifying the particular pathogen strain or species is critical for implementing effective patient management or treatment strategies.

Molecular diagnostics, such as the polymerase chain reaction (PCR) assay, are the gold standard for identifying the specific pathogen. PCR can detect nucleic acid biomarkers of multiple pathogens from a single patient specimen with high sensitivity and specificity. The number of distinct pathogens that can be detected in parallel from a sample is limited by a number of different factors, such as the volume of the sample, cost of running multiple PCR assays, and the number of detection channels that can be differentiated.

### SUMMARY

Described herein is a method for increasing the number of distinct pathogens that can be detected in parallel from a single sample using a standard PCR assay. The method uses fluorescent compounds that are multiplexed in a common spectral channel utilizing properties of the fluorescent compounds themselves, which can allow for a significantly higher throughput of molecular diagnostic results without need for a larger volume of a sample, increased costs, or increased number of differentiated detection channels.

In an aspect, the present disclosure can include a method for multiplexing fluorescent compounds in a common spectral channel to determine a presence or absence of unique testable conditions that includes the following steps. Adding at least two fluorescent compounds, each designed to have a peak fluorescence excitation and/or emission within a common spectral channel, to a sample in a tube, wherein each of the at least two fluorescent compounds is used to test for a unique testable condition. Performing a polymerase chain reaction (PCR) assay on contents of the tube to create a PCR product. And, determining a spectral channel signal of each of the at least two fluorescent compounds based on at least two properties of each of the at least two fluorescent compounds, where the at least two properties of each of the at least two fluorescent compounds comprise at least a property at a physical testing condition and another property at another physical testing condition. The property of each of the at least two fluorescent compounds being different for each of the at least two fluorescent compounds at the physical testing condition and the other property of each of the at least two fluorescent compounds being different for each of the at least two fluorescent compounds at the other physical testing condition. The spectral channel signals of the at least two fluorescent compounds are analyzed to indicate a presence or absence of each of the unique testable conditions.

### BRIEF DESCRIPTION OF THE DRAWINGS

The foregoing and other features of the present disclosure will become apparent to those skilled in the art to which the present disclosure relates upon reading the following description with reference to the accompanying drawings, in which:

FIG. 1 includes graphical examples of properties of one or more fluorescent compounds;

FIGS. 2 and 3 are a process flow diagram illustrating a method for increasing the number of distinct testable conditions that can be detected in parallel from a sample; and

FIG. 4 is a diagram of a system that can be used to execute the methods of FIGS. 2 and 3.

### DETAILED DESCRIPTION

### I. Definitions

Unless otherwise defined, all technical terms used herein have the same meaning as commonly understood by one of ordinary skill in the art to which the present disclosure pertains.

As used herein, the singular forms “a,” “an” and “the” can also include the plural forms, unless the context clearly indicates otherwise.

As used herein, the terms “comprises” and/or “comprising,” can specify the presence of stated features, steps, operations, elements, and/or components, but do not preclude the presence or addition of one or more other features, steps, operations, elements, components, and/or groups.

As used herein, the term “and/or” can include any and all combinations of one or more of the associated listed items.

As used herein, the terms “first,” “second,” etc. should not limit the elements being described by these terms. These terms are only used to distinguish one element from another. Thus, a “first” element discussed below could also be termed a “second” element without departing from the teachings of the present disclosure. The sequence of operations (or acts/steps) is not limited to the order presented in the claims or figures unless specifically indicated otherwise.

As used herein, the term “Polymerase Chain Reaction (PCR) assay” refers to quantitative PCR, which is an established tool for DNA quantification that measures the accumulation of DNA product after each round of PCR amplification using fluorescent compounds. PCR is in vitro technique for rapidly synthesizing large quantities of a given DNA segment that involves separating the DNA into its two complementary strands, using DNA polymerase to synthesize two-stranded DNA from each single strand, and repeating the process. This process results in the duplication of the original DNA, with each of the new molecules containing one old and one new strand of DNA. Then each of these strands can be used to create two new copies, and so on, and so on. The cycle of denaturing and synthesizing new DNA is repeated as many as 30 or 40 times, leading to more than one billion exact copies of the original DNA segment.

As used herein, the terms “well” and “tube” or the like refer to a single vessel for testing a sample. A well plate, also known as a microplate, multiwell, etc., is a flat plate comprising multiple wells used to test a plurality of samples through the same assay, such as a PCR assay, at one time.

As used herein, the term “fluorescent compound” refers to a chemical compound that can absorb light energy at specific wavelengths and re-emit light at longer wavelengths. A fluorescent compound can bind to a diagnostic target of interest and the presence of the diagnostic target can be measured based on the known excitation and emission spectra of the fluorescent compounds, particularly the peak excitation and emissions wavelengths. An example of a fluorescent compound is a fluorescent dye.

As used herein, the term “fluorescent emission” refers to the wavelength(s) of light emitted after a fluorescent compound has absorbed light energy at a given wavelength.

As used herein, the term “fluorescent excitation” refers to the wavelength(s) of light that can be absorbed so as to emit a single emission wavelength.

As used herein, the term “peak fluorescent excitation” refers to the wavelength of light that most efficiently excites a fluorescent compound, e.g., is absorbed the most efficiently

As used herein, the term “peak fluorescent emission” refers to the wavelength of light that a fluorescent compound is most likely to emit for a given excitation wavelength.

As used herein, the term “spectral channel” refers to distinguishable wavelength ranges of the light spectrum, such as wavelength ranges that correspond to different ranges of visible light (e.g., corresponding to one or more of red light, orange light, green light, cyan light, blue light, and violet light). The visual light spectrum is used as a non-limiting example. A common spectral channel refers to one of these wavelength ranges where multiple spectral channel signals are measured.

As used herein, the term “spectral channel signal” refers to a contribution of a given fluorescent compound to a fluorescent emission measurement.

As used herein, the term “unique testable condition” refers to a target that can be identified by fluorescent measurements during and/or after a PCR assay. Each fluorescent compound added to the sample can be used to determine a different unique testable condition.

As used herein, the term “malady” refers to a disease, disorder, illness, or injury that has one or more symptoms that can be ameliorated and/or treated with stimulation of one or more neural elements. Each malady can be determined based on a unique testable condition of a source of the malady (e.g., a pathogen a DNA target, an RNA target, a single nucleotide polymorphism (SNP), a mutation, or a genotype).

As used herein, the term “physical testing condition” refers to an environmental condition that can be altered between fluorescent measurements. Non-limiting examples of physical testing conditions include: temperature, photobleaching, excitation light wavelength and/or intensity, electric charge, or pH.

As used herein, the term “property” refers to an inherent characteristic of a fluorescent compound that can based on the fluorescent compounds structure and/or environment. Non-limiting examples of properties include how a fluorescent compound responds to a given temperature, pH, electric charge, wavelength of light, light intensity, or the like. Properties can be common to more than one fluorescent compound or can be unique to a specific fluorescent compound. A unique property is a property that a given fluorescent compound does not share with other fluorescent compounds in the same testing sample.

As used herein, the term “multiplex” refers to combining multiple measurable analytes (such as multiple fluorescent compounds) in a single assay or experiment to increase the number of results from a single sample.

### II. Overview

Molecular diagnostics, such as the polymerase chain reaction (PCR) assay, are the gold standard for syndromic testing. The detection of nucleic acid biomarkers of multiple pathogens can be carried out from a single patient specimen using PCR with high sensitivity and specificity. This is made possible because PCR differentiates pathogens by detecting short and distinct sequences of RNA or DNA using nucleic acid “primers” that bind to the genetic material that uniquely corresponds to that pathogen. Within the small reaction vessel used for PCR, a mixture of enzymes and molecules including these primers replicate the short sequence targets to the scale of billions of copies, which are detected in real time using molecular probes. These molecular probes (called hydrolysis probes, TaqMan probes, or molecular beacons) are labeled with fluorescent dyes, or fluorophores, which can be detected using optical devices.

One PCR testing format is to perform parallel reactions in separate reaction tubes, where each tube is detecting a different pathogen target. In this design, the number of pathogens that can be detected is limited by the volume of the sample, which is split into multiple tubes, as well as the number of available tube positions within the instrument and the reagent costs associated with running multiple independent reactions. This is the format that is used by the very successful BioFire FilmArray system, which performs a respiratory pathogen panel (RPP) detection assay for 23 pathogen targets by splitting the sample into 23 independent relations. The downside of this assay is of course cost, as it is one of the most expensive respiratory diagnostic tests available. To mitigate these limitations, another common format combines the reagents for detecting each pathogen into a single tube, where the individual targets are generally distinguished using fluorescent dyes. The widely used Thermo Fisher Scientific QuantStudio 5 instrument, which was one of the first instruments to receive FDA EUA for multiplexed Covid-19/Influenza testing, uses this approach. However, current instrumentation is limited to the detection of 5-6 independent targets because there is only 5-6 fluorescent channels with sufficient spectral separation to distinguish them from one another. To address the limitation available detection “channels,” we have developed a method for differentiating two or more fluorescent dyes with significant spectral overlap (i.e., similar excitation and emission peaks).

The present disclosure improves on the PCR format of combining reagents for detect pathogens into a single tube and distinguishing the individual targets using fluorescent compounds. Traditionally, as described above, the number of individual targets that can be identified is limited by the number of fluorescent compounds that can be distinguished at peak excitation and/or emission. The present disclosure describes a method that multiplexes fluorescent compounds in a common spectral channel so that the number of individual targets are not limited only by the number of dyes that can be distinguished at peak excitation and/or emission. By utilizing multiple fluorescent compounds that have a common spectral channel for peak excitations and/or emission, but are structurally different, the present method, utilizes properties unique to each of the fluorescent compounds to further differentiate them. Each unique property can be leveraged to gain a unique measurement. For example, if there are six fluorescent compounds with unique structures and unique properties in a common spectral channel, then the present method can distinguish six individual targets where the traditional method can only distinguish one. Thus, a single PCR sample can be tested for significantly more targets without requiring a larger volume of sample, more costly equipment, or new fluorescent compounds.

### III. Utilizing Properties of Fluorescent Compounds

The most commonly utilized property of fluorescent compounds is that they fluoresce in response to light, i.e., they absorb light energy at specific wavelengths and re-emit light at longer wavelengths. Fluorescent compounds are often grouped by what colors of light they excite at and what colors of light they emit at. Many forms of medical and scientific testing utilize fluorescence to help with quantifying targets in test samples. However, fluorescent compounds also have many other properties that are under-utilized, such as, but not limited to, how they respond to environmental conditions (such as temperature, pH, light intensity, and electric charge), quantum efficiency, fluorescence lifetime, and emission and excitation at non-peak wavelengths. These properties can create measurable differences in the fluorescence emission of the fluorescence compounds. Measurements can be done during and/or after an assay has been run, depending on the one or more other properties that are being exploited. Many fluorescent compounds that are normally indistinguishable based only on peak excitation and/or emission readings can be differentiated based on these measurable differences. Fluorescent compounds to be added to a single sample can be chosen so that while their peak excitation and/or emission readings are the same, their other properties are unique and can be used to identify the presence or absence of different targets in the sample. These properties can be detected either during a PCR or after the PCR.

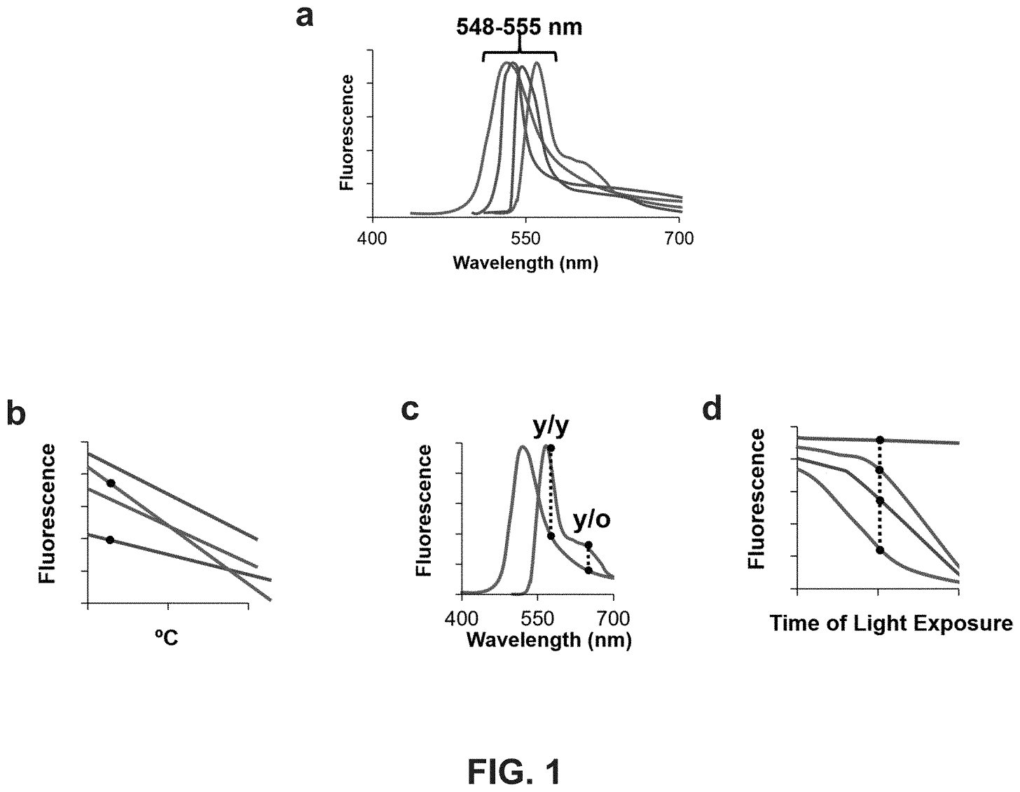

Increasing the number of unique testable conditions that can be detected in parallel from a sample in a single PCR well hinges on each of the fluorescent compounds having a number of unique, measurable properties. FIGS. 1a-d illustrate graphical examples of how a number of fluorescent compounds can have at least one common property and at least one other property that is unique for a given physical testing condition. It should be understood that a number of different other properties may be exhibited by certain fluorescent compounds.

FIG. 1, element a, shows a graphical example of four commercially available fluorescent compounds that have similar peak excitation and emission properties in the yellow common spectral channel. The four fluorescent compounds graphed are ATTO, HEX, YAK, and MAX, however this is just an example and other fluorescent compounds can be used. Each fluorescent compound can be associated with a unique testable condition and used for multiplexing within the yellow common spectral channel. An increase in a spectral channel signal in the yellow common spectral channel indicates that at least one of the unique testable conditions is present. The properties unique to each of the fluorescent compounds can then be utilized to determine which of the one or more unique testable conditions is present. Most fluorescent compounds have differences in their properties due to differences in structures. Unique properties for each of the fluorescent compounds can include, but are not limited to, quantum yields, response to temperature (shown in FIG. 1, element b), difference in emission spectra at a given excitation wavelength (shown for two fluorescent compounds in FIG. 1, element c), and sensitivity to light exposure over time (shown in FIG. 1, element d). Fluorescence emission measurements can be made with physical testing conditions altered to match the physical testing conditions where the fluorescent compounds properties differ.

### IV. Methods

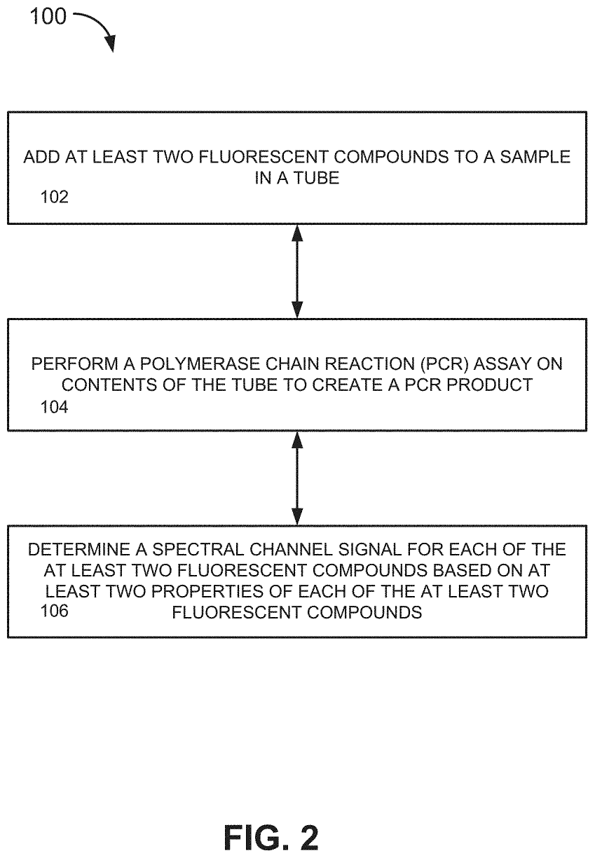

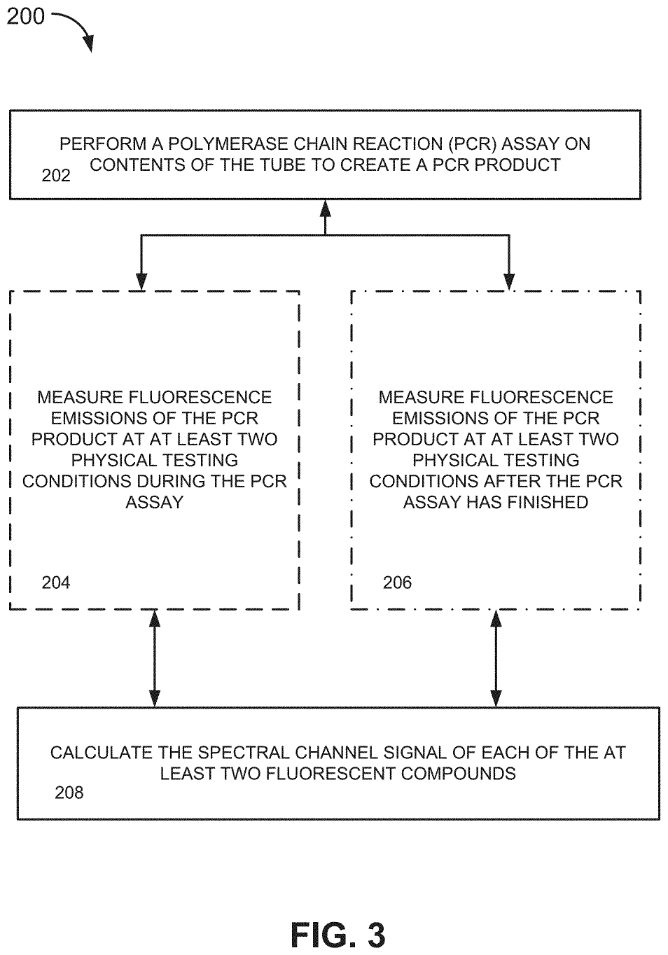

An aspect of the present disclosure can include methods 100 and 200, shown in FIGS. 2 and 3, for increasing the number of unique testable conditions that can be detected in parallel from a sample in a single PCR well. The method uses fluorescent compounds that are multiplexed in a common spectral channel utilizing properties of the fluorescent compounds themselves, which can allow for a significantly higher throughput of molecular diagnostic results without need for a larger volume of a sample, increased costs, or increased number of differentiated detection channels.

For purposes of simplicity, the methods 100 and 200 are shown and described as being executed serially; however, it is to be understood and appreciated that the present disclosure is not limited by the illustrated order as some steps could occur in different orders and/or concurrently with other steps shown and described herein. Moreover, not all illustrated aspects may be required to implement the methods 100 and 200, nor are methods 100 and 200 limited to the illustrated aspects.

Referring now to FIG. 2, illustrated is a method 100 for increasing the number of unique testable conditions that can be detected in parallel from a sample in a single PCR well. At 102, at least two fluorescent compounds can be added to a sample in a tube (e.g., a PCR well), where each fluorescent compound can test for a unique testable condition. In response to the presence of particular RNA or DNA sequence, the fluorescence increases. Alternatively, each of the at least two fluorescent compounds can attach to a reagent to form a dyed reagent constructed to attached to a specific target. The dyed reagents can then be added to the tube to attach to each of the targets. Each of the at least two fluorescent compounds added to the tube can be structural different but designed to have a peak fluorescence excitation and/or emission within a common spectral channel. For example, the peak fluorescence excitation and/or emission of the at least two fluorescent compounds within the common color spectral channel can be within +/−20 nm, +/−15 nm, or +/−10 nm of each other. Examples of common spectral channels used with a PCR assay include, but are not limited to: a blue channel, a green channel, a yellow channel, an orange channel, a red channel, or a crimson channel. For example, the common spectral channel can be the yellow channel and the at least two fluorescent compounds can be at least two of: HEX, MAX, SUN, JOE, ATTO 532, and YAKIMA Yellow. Each of the at least two fluorescent compounds added to the sample can be used to test for a unique testable condition, which corresponds to a source of a potential malady of the patient the sample was taken from. The sources of the maladies being tested for can be, but are not limited to, a pathogen, a DNA target, an RNA target, a single nucleotide polymorphism (SNP), a mutation, or a genotype.

At 104, a PCR assay can be performed on the contents of the tube to create a PCR product based on the sample. As is known in the field, PCR is an iterative process by which the DNA of the sample is amplified. The PCR product can be an intermediate product formed during the PCR assay process. The PCR product can also be a final product formed when the PCR assay is completed. In other words, fluorescence emissions produced by the presence of the RNA or DNA target can be measured during and/or after the PCR assay has finished.

At 106, a spectral channel signal of each of the at least two fluorescent compounds can be determined based on at least two properties of the at least two fluorescent compounds. The at least two properties of each of the at least two fluorescent compounds can include a property at a physical testing condition and another property at another physical testing condition. Each of the at least two physical testing conditions can be determined based on a sensitivity analysis. While only two physical testing conditions are described herein, it should be understood that any number of physical testing conditions can exist, as long as the number of physical testing conditions is equal to or greater than the number of fluorescent compounds. The property of each of the at least two fluorescent compounds is different for each of the at least two fluorescent compounds at the physical testing condition and the other property of each of the at least two fluorescent compounds is different for each of the at least two fluorescent compounds at the other physical testing condition. The spectral channel signals of the at least two fluorescent compounds can be analyzed to indicate a presence or absence of each of the unique testable conditions. In one example, when the at least two fluorescent compounds added to the sample in the tube is six fluorescent compounds, each designed to have a peak fluorescence excitation and/or emission in the common spectral channel, added to the sample in the tube, then determining further comprises determining six spectral channel signals each representative of one of the six fluorescent compounds.

In another example, when the at least two fluorescent compounds are a first fluorescent compound and a second fluorescent compound. The first fluorescent compound can respond to a first physical testing condition, such as a given temperature (for example 60° F.), in a first manner and the second fluorescent compound can respond to the first physical testing condition, the given temperature, in a second manner. The different responses can be measured by measuring the fluorescence emissions of a sample of the first fluorescent compound and a sample of the second fluorescent compound when the first physical testing condition is altered but no other environmental factors are altered. At the second physical testing condition, such as a different temperature for example 95° F., the first fluorescent compound can respond in a third manner and the second fluorescent compound can respond in a fourth manner. Again, the different responses at the second physical testing condition can be measured by measuring the fluorescence emissions of a sample of the first fluorescent compound and a sample of the second fluorescent compound when the second physical testing condition is altered but no other environmental factors are altered. These measurements are physical constants that are unique for each physical testing condition and fluorescent compound.

The at least two properties of each of the at least two fluorescent compounds are unaffected by the presence of the others of the at least two fluorescent compounds in the tube. Additionally, the at least two properties of each of the at least two fluorescent compounds do not change when two or more of the fluorescent compounds are mixed together in the sample or when the PCR product is formed. The at least two properties comprise at least two of: a response to a temperature, a response to photobleaching, a response to a pH, or a response to an electric charge, a quantum efficiency, a fluorescence lifetime, and a measure of fluorescence intensity differences of one of the fluorescent compounds between different wavelengths of the emission spectra.

Referring now to FIG. 3, which shows more detailed steps on how the at least two fluorescent compounds can be multiplexed to increase the number of targets that can be identified in a single sample. At 202, the PCR assay can be performed on the contents of the tube to create a PCR product as described in greater detail above. At 204 and 206, fluorescence emissions of the PCR product at the at least two physical testing conditions can be measured during and/or after the PCR assay has finished. The at least two measurements can be done only during the PCR assay, on an intermediate PCR product; only after the PCR assay has finished, on a completed PCR product; or any combination thereof to have at least as many measurements as fluorescent compounds that were added to the sample. Non-limiting examples of fluorescence emission measurements that can be done during and/or after the PCR assay has finished include: a fluorescence intensity under standard PCR conditions; a fluorescence intensity at a given temperature, wherein the given temperature is not one of the standard PCR conditions; a fluorescence lifetime, wherein the fluorescence lifetime is a fluorescence intensity measured immediately after the excitation has turned off; and a fluorescence intensity at two or more emission wavelengths, wherein the two or more emission wavelengths are not one of the standard PCR conditions. Non-limiting examples of fluorescence emission measurements that can be done after the PCR assay has finished include: a difference in fluorescence intensity at peak emission at two different times during the PCR assay, a slope of the fluorescence intensity decrease at peak emission when heating the PCR product from a first temperature to a second temperature, a difference in fluorescence intensity at peak emission before and after photobleaching the PCR product, a difference in fluorescence intensity at peak emission before and after changing the pH of the PCR product, a difference in fluorescence intensity at peak emission before and after applying an electric charge to the PCR product, and a difference in fluorescence intensity of the PCR product at two or more emission wavelengths.

At 208, the spectral channel signal of each of the at least two fluorescent compounds can be calculated based on the at least two fluorescence emission measurements of the PCR product at the at least two physical testing conditions. The calculations are also based on known physical constants for each of the at least two fluorescent compounds added to the sample at each of the at least two physical testing conditions. The physical constants are the fluorescence emission measurements of each the at least two fluorescent compounds at each of the at least two physical testing conditions. The spectral channel signal of each of the at least two fluorescent compounds can be determined by solving a series of linear equations represented by a matrix of the fluorescence emission measurements of the PCR product multiplied by the inverse of a matrix of the physical constants for each of the at least two fluorescent compounds at each of the at least two physical testing conditions.

For example, referring back to the example with the first and second fluorescent compounds, the first the second fluorescent compounds can be HEX and ATTO, respectively. The HEX and ATTO fluorescent compounds are added to the sample in the tube and the PCR assay is performed to create a PCR product, then fluorescence emission measurements of the PCR product can be made at the same first and second physical testing conditions described above. The spectral channel signal of the first fluorescent compound, HEX, (X_{Hex}) and the spectral channel signal of the second fluorescent compound, ATTO, (X_{Atto}) can be determined based on the physical constants that represent the properties of HEX and ATTO at the first and second physical testing conditions (α_{Hex Phys. Prop. 1,2} and α_{Atto Phys. Prop. 1,2}) and the fluorescence emission measurements of the PCR product at the first and second physical testing conditions (M_{Phys. Prop. 1,2}). Shown in linear equations below:

$$[ \begin{matrix}{M}_{Phys . Prop . 1} \\ {M}_{Phys . Prop . 2}\end{matrix} ] = [ \begin{matrix}{α}_{Phys . Prop . 1} & {α}_{Atto Phys . Prop . 1} \\ {α}_{Phys . Prop . 2} & {α}_{Atto Phys . Prop . 2}\end{matrix} ] [ \begin{matrix}{X}_{Hex} \\ {X}_{Atto}\end{matrix} ]$$

Referring again to the general calculation step at 208, the at least two fluorescence emission measurements can be represented as an N×1 matrix, where N is the number of fluorescence emission measurements that is at least two. In matrix form, the matrix of the at least two fluorescence emissions measurements is composed of an N×N matrix of physical constant measurements of each of the at least two fluorescent compounds at each of the at least two physical testing conditions and an N×1 matrix of the spectral channel signal of each of the at least two fluorescent compounds. Like the example linear equation shown above where N=2. N represents the number of the at least two fluorescent compounds, the number of spectral channel signals to be solved for, the number of unique testable conditions, the number of physical testing conditions, and the number of unique physical constants for each fluorescent compound. Calculating the spectral channel signal of each the at least two fluorescent compounds requires solving a linear matrix function of the N×1 matrix of the at least two fluorescence emission measurements times the inverse of the N×N matrix of physical constant measurements of each of the at least two fluorescent compounds at each of the at least two physical testing conditions. the linear algebra matrix function comprises a series of at least two linear equations, wherein the number of linear equations is equal to or greater than the number of the at least two physical testing conditions.

Based on the calculated spectral channel signal of each of the at least two fluorescent compounds in the measured fluorescence emissions of the PCR product the presence or absence of unique testable conditions can be determined. Recall, that each fluorescent compounds can tag one unique testable condition. Therefore, if one or more spectral channel signals is zero or near zero, then it can be determined that the target unique testable condition is not found in that sample. Other values of spectral channel signals can mean the presence, partial presence, or absence of one or more unique testable conditions in a sample. In this way the number of distinct testable conditions that can be detected in parallel from a single sample is increased.

### V. Systems

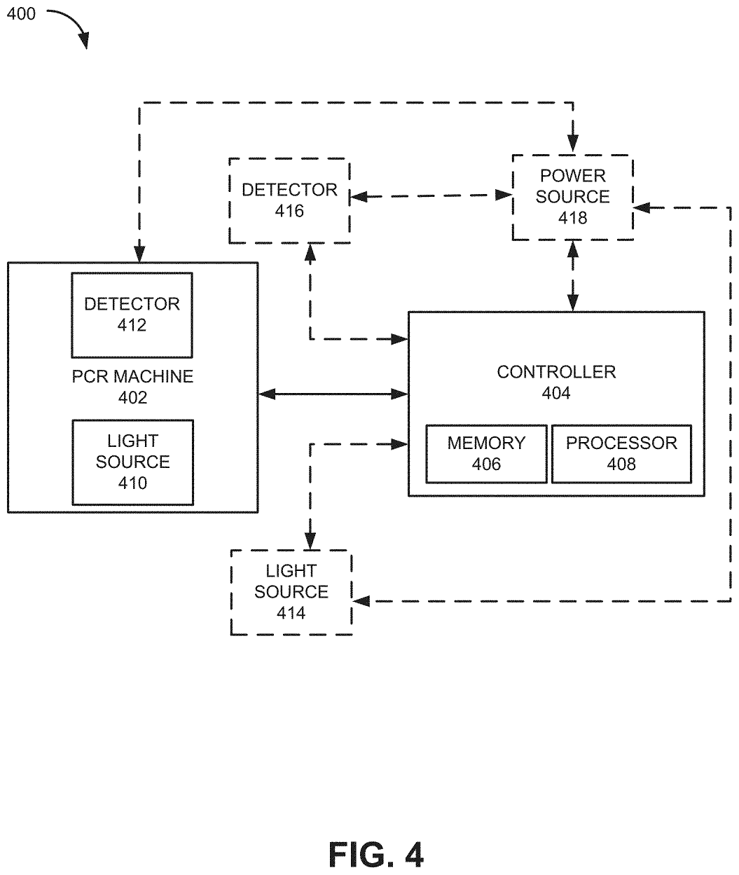

An example system (system 400 shown in FIG. 4) can be used for performing part or all of the method described above for increasing the number of distinct testable conditions that can be detected in parallel from a single sample. The system can include at least a PCR machine 402, comprising a well plate with at least one well for a sample (not illustrated), and a controller 404 (which can be, for example, part of the PCR machine (not shown), external to the PCR machine (as shown), or a combination (now shown)) that has a memory 406 and a processor 408. The PCR machine can also include a light source 410 and a detector 412. Additionally or alternatively, the system can also include a separate light source 414, a separate detector 416, and a power source 418 in electrical communication with the controller 404. In some instances, the controller 404, the PCR machine 402, and/or the light source 416 and detector 418 can be electrically coupled to the power supply 418. In other instance one or more of the controller 404, the PCR machine 401 and/or the light source 416 and detector 418 can have an internal power source (e.g., a battery).

The controller 404 can be configured to generate and transmit a control signal to at least one of the PCR machine 402, the separate light source 414, and the separate detector 416 via a wired connection and/or a wireless connection. The controller 404 can be a computing device (e.g., a general-purpose computer, special purpose computer, and/or other programmable data processing apparatus) that can include or be otherwise associated with a non-transitory memory 406 storing instructions (e.g., computer program instructions) that, upon execution by a processor 408, can create a mechanism for implementing the functions of the controller (e.g., performing the PCR assay, measuring fluorescence emissions, calculating, etc.). The controller 404 can control at least one of the performance of the PCR assay, the measuring of the fluorescence emissions, the calculations of the spectral channel signal of each of the at least two fluorescent compounds, and analyzing the spectral channel signals to determine the presence or absence of one or more unique testable conditions.

The system 400 can be configured so that the at least two physical testing conditions can be easily altered. The controller 404 can optionally control the physical testing conditions utilizing at least one of the PCR machine 402, the light source 410 and/or 414, the power source 418, or additional components, to affect the properties of the at least two fluorescent compounds added to the sample. For example, properties of the fluorescent compounds comprise: a response to temperature, a response to photobleaching, a response to pH, or a response to electric charge, a quantum efficiency, a fluorescence lifetime, and a measure of fluorescence intensity differences of one of the fluorescent compounds between different wavelengths of the emission spectra. The controller 404, via the light detector 412 and/or 416, can measure the fluorescence emission response of the PCR product during and/or after the PCR assay has finished depending on the type of measurement. For example, during and/or after the PCR assay the controller 404, and light detector 412 and/or 416, can be configured to measure at least one of: a fluorescence intensity under standard PCR conditions; a fluorescence intensity at a given temperature, wherein the given temperature is not one of the standard PCR conditions; a fluorescence lifetime, wherein the fluorescence lifetime is a fluorescence intensity measured immediately after the excitation has turned off; and a fluorescence intensity at two or more emission wavelengths, wherein the two or more emission wavelengths are not one of the standard PCR conditions. In another example, after the PCR assay has finished, the controller, and the light detector can be configured to measure at least one of: a difference in fluorescence intensity at peak emission at two different times during the PCR assay, a slope of the fluorescence intensity decrease at peak emission when heating the PCR product from a first temperature to a second temperature, a difference in fluorescence intensity at peak emission before and after photobleaching the PCR product, a difference in fluorescence intensity at peak emission before and after changing the pH of the PCR product, a difference in fluorescence intensity at peak emission before and after applying an electric charge to the PCR product, and a difference in fluorescence intensity of the PCR product at two or more emission wavelengths. Based on at least two of the above measurements the controller can be configured to calculate spectral channel signal of each of the at least two fluorescent compounds and to determine the presence or absence of unique testable conditions.

From the above description, those skilled in the art will perceive improvements, changes, and modifications. Such improvements, changes and modifications are within the skill of one in the art and are intended to be covered by the appended claims.

The following is claimed:

1. A method comprising:
adding at least two fluorescent compounds, each designed to have a peak fluorescence excitation and/or emission within a common spectral channel, to a sample in a tube, wherein each of the at least two fluorescent compounds is used to test for a unique testable condition;
performing a polymerase chain reaction (PCR) assay on contents of the tube to create a PCR product; and
determining a spectral channel signal of each of the at least two fluorescent compounds based on at least two properties of each of the at least two fluorescent compounds,
wherein the at least two properties of each of the at least two fluorescent compounds comprise at least a property at a physical testing condition and another property at another physical testing condition,
wherein the property of each of the at least two fluorescent compounds is different for each of the at least two fluorescent compounds at the physical testing condition and the other property of each of the at least two fluorescent compounds is different for each of the at least two fluorescent compounds at the other physical testing condition, and
wherein the spectral channel signals of the at least two fluorescent compounds are analyzed to indicate a presence or absence of each of the unique testable conditions.

2. The method of claim 1, wherein each of the unique testable conditions corresponds to a presence of a known RNA or DNA sequence.

3. The method of claim 2, wherein the known RNA or DNA sequence is one of: a pathogen, a DNA target, an RNA target, a single nucleotide polymorphism (SNP), a mutation, or a genotype.

4. The method of claim 1, wherein when six fluorescent compounds, each designed to have a peak fluorescence excitation and/or emission in the common spectral channel, are added to the sample in the tube, the determining further comprises determining six spectral channel signals each representative of one of the six fluorescent compounds.

5. The method of claim 1, wherein the at least two fluorescent compounds are structurally different.

6. The method of claim 1, wherein the at least two properties comprise at least two of: a response to a temperature, a response to photobleaching, a response to a pH, or a response to an electric charge, a quantum efficiency, a fluorescence lifetime, and a measure of fluorescence intensity differences of one of the fluorescent compounds between different wavelengths of the emission spectra.

7. The method of claim 1, wherein adding the at least two fluorescent compounds to the sample in the tube further comprises:
attaching each of the at least two fluorescent compounds to a reagent to form a dyed reagent; and
adding the dyed reagents to the tube.

8. The method of claim 1, wherein the common spectral channel is one of a blue channel, a green channel, a yellow channel, an orange channel, a red channel, or a crimson channel.

9. The method of claim 1, wherein the common spectral channel is a yellow channel and the at least two fluorescent compounds are at least two of: HEX, MAX, SUN, JOE, ATTO 532, and YAKIMA Yellow.

10. The method of claim 1, wherein the at least two properties of each of the at least two fluorescent compounds are unaffected by the presence of the others of the at least two fluorescent compounds in the tube.

11. The method of claim 1, further comprising measuring fluorescence emissions of the PCR product at the at least two physical testing conditions during and/or after the PCR assay has finished.

12. The method of claim 11, wherein the spectral channel signal of each of the at least two fluorescent compounds are calculated based on the at least two fluorescence emission measurements of the PCR product at the at least two physical testing conditions.

13. The method of claim 12, wherein the at least two fluorescence emission measurements are represented as an N×1 matrix, where N is the number of fluorescence emission measurements that is at least two.

14. The method of claim 13, wherein in matrix form, the matrix of the at least two fluorescence emissions measurements is composed of an N×N matrix of physical constant measurements of each of the at least two fluorescent compounds at each of the at least two physical testing conditions and an N×1 matrix of the spectral channel signal of each of the at least two fluorescent compounds.

15. The method of claim 13, wherein the N also represents the number of the at least two fluorescent compounds and the number of the spectral channel signals.

16. The method of claim 14, wherein the determining the spectral channel signal of each the at least two fluorescent compounds further comprises solving a linear matrix function of the N×1 matrix of the at least two fluorescence emission measurements times the inverse of the N×N matrix of physical constant measurements of each of the at least two fluorescent compounds at each of the at least two physical testing conditions.

17. The method of claim 16, wherein the linear algebra matrix function comprises a series of at least two linear equations, wherein the number of linear equations is equal to or greater than the number of the at least two physical testing conditions.

18. The method of claim 17, wherein the measured fluorescence emissions comprise at least two of:
a fluorescence intensity under standard PCR conditions;
a fluorescence intensity at a given temperature, wherein the given temperature is not one of the standard PCR conditions;
a fluorescence lifetime, wherein the fluorescence lifetime is a fluorescence intensity measured immediately after the excitation has turned off;
a fluorescence intensity at two or more emission wavelengths, wherein the two or more emission wavelengths are not one of the standard PCR conditions;
a difference in fluorescence intensity at peak emission at two different times during the PCR assay;
a slope of the fluorescence intensity decrease at peak emission when heating the PCR product from a first temperature to a second temperature;
a difference in fluorescence intensity at peak emission before and after photobleaching the PCR product;
a difference in fluorescence intensity at peak emission before and after changing the pH of the PCR product;
a difference in fluorescence intensity at peak emission before and after applying an electric charge to the PCR product; and
a difference in fluorescence intensity of the PCR product at two or more emission wavelengths.

19. The method of claim 1, wherein the at least two physical testing conditions are determined based on a sensitivity analysis.

20. The method of claim 1, wherein the peak fluorescence excitation and/or emission of the at least two fluorescent compounds within the common color spectral channel are within +/−20 nm, +/−15 nm, or +/−10 nm of each other.
